(19) United States
(12) Patent Application Publication
(10) Pub. No.: US 2026/0167458 A1
(43) Pub. Date: Jun. 18, 2026

(54) ELEVATING APPARATUS FOR MOBILITY VEHICLE AND BUILDING HAVING THE SAME

(71) Applicants: Hyundai Motor Company, Seoul (KR); Kia Corporation, Seoul (KR)

(72) Inventors: Dae Young GWON, Hwaseong-si (KR); Ji Hyun SONG, Hwaseong-si (KR)

(73) Assignees: Hyundai Motor Company, Seoul (KR); Kia Corporation, Seoul (KR)

(21) Appl. No.: 19/225,686

(22) Filed: Jun. 2, 2025

(30) Foreign Application Priority Data
Dec. 16, 2024 (KR) 10-2024-0187450

(51) Int. Cl. B66B 11/00; A61L 2/10; A61L 9/20; B08B 3/02; B08B 13/00; B60S 3/04; B66B 11/02

(52) U.S. Cl. CPC B66B 11/006; A61L 2/10; A61L 9/20; B08B 3/024; B08B 13/00; B60S 3/04; B66B 11/0226; B66B 11/0233; A61L 2202/11; A61L 2202/17; A61L 2209/12

(57) ABSTRACT

In an elevating apparatus for a mobility vehicle and a building having the same, a vehicle washing function is provided in an elevator so that the mobility vehicle is washed for a time period for which the mobility vehicle is moved by the elevator and the time and costs required to wash the mobility vehicle are reduced, a foothold function is provided to a passenger who gets in or out of the mobility vehicle so that convenience related to a situation in which the passenger gets in or out of the mobility vehicle is ensured, a clean state in the elevator is maintained, a sense of luxury is provided to the passenger, who utilizes the elevator, by implementing various lighting functions in the elevator, and a boarding or alighting direction for the passenger or an exiting or entering direction for the mobility vehicle is guided by lighting.

### CROSS-REFERENCE TO RELATED APPLICATION

The present application claims priority to Korean Patent Application No. 10-2024-0187450, filed on Dec. 16, 2024, the entire contents of which are incorporated herein for all purposes by this reference.

### BACKGROUND OF THE DISCLOSURE

### Field of the Disclosure

The present disclosure relates to an elevating apparatus for a mobility vehicle and a building having the same, the elevating apparatus being configured to move a mobility vehicle to various floor levels by an elevator in a building.

### Description of the Related Art

Future mobility vehicles serve as transportation means and provide various services while providing occupancy spaces for passengers. As functions of the mobility vehicles are diversified, the mobility vehicles are accommodated in buildings to extend convenience. Therefore, buildings in which mobility vehicles may be parked are also being variously developed.

An elevating apparatus is provided in a building capable of accommodating a mobility vehicle and serves to move the mobility vehicle to a floor level.

The elevating apparatus has a space capable of accommodating the mobility vehicle or a passenger. The elevating apparatus moves the mobility vehicle or the passenger to a particular floor level by moving to the floor level in the building.

However, because the elevating apparatus simply operates to move the mobility vehicle or the passenger without providing other services, a time period for which the elevating apparatus moves is wasted.

In addition, the elevating apparatus, in which the mobility vehicle is accommodated, has a large internal space to accommodate the mobility vehicle, but the large internal space is vulnerable to contamination, which may cause unpleasantness to the passenger who uses the elevating apparatus.

The foregoing explained as the background is intended merely to aid in the understanding of the background of the present disclosure, and is not intended to mean that the present disclosure falls within the purview of the related art that is already known to those skilled in the art.

### SUMMARY OF THE DISCLOSURE

The present disclosure is proposed to solve these problems and aims to provide a vehicle washing function in an elevator to wash a mobility vehicle for a time period for which the mobility vehicle is moved by the elevator, reducing the time and costs required to wash the mobility vehicle.

The present disclosure also aims to provide a foothold function to a passenger who gets in or out of a mobility vehicle, allowing the passenger to conveniently get in or out of the mobility vehicle.

The present disclosure also aims to maintain a clean state in an elevator, provide a sense of luxury to a passenger, who utilizes the elevator, by implementing various lighting functions in the elevator, and ensure elevator usage satisfaction by guiding a boarding or alighting direction for a passenger or an exiting or entering direction for a mobility vehicle by providing lighting.

Technical problems to be solved by the present disclosure are not limited to the above-mentioned technical problems, and other technical problems, which are not mentioned above, may be clearly understood from the following descriptions by those skilled in the art to which the present disclosure pertains.

To achieve the above-mentioned objects, the present disclosure provides an elevating apparatus for a mobility vehicle, the elevating apparatus including: an elevator provided in a structure, configured to be movable upward or downward, including an accommodation space configured to accommodate a mobility vehicle, and including at least one opening portion; a base plate provided in the elevator, configured to be rotatable on a bottom surface of the accommodation space, and configured to align an entrance/exit of the mobility vehicle or a traveling direction of the mobility vehicle with the opening portion while rotating in a state in which the mobility vehicle is loaded; and a multifunctional plate provided in the elevator and configured to move in an upward/downward direction in the accommodation space, the multifunctional plate being configured to cover the base plate between the entrance/exit of the mobility vehicle and the opening portion of the elevator while moving downward to provide a foothold function to a passenger.

A contact portion may be provided at a center portion of the base plate, a connection portion configured to come into contact with the contact portion may be provided in a lower portion of the mobility vehicle, and the connection portion may come into contact with the contact portion and is guided when the mobility vehicle enters the base plate so that a position of the mobility vehicle is aligned with the center portion of the base plate.

The connection portion may extend so that a width thereof gradually decreases from a lower rim of the mobility vehicle toward a center portion of the mobility vehicle, and the contact portion and the connection portion may guide the mobility vehicle so that the mobility vehicle is positioned at the center portion of the base plate when the mobility vehicle enters the base plate.

A door portion may be provided in an inlet of the connection portion of the mobility vehicle, and the door portion may be opened when the mobility vehicle enters the base plate.

The base plate may rotate after the position of the connection portion of the mobility vehicle is aligned with the center portion of the base plate by the contact portion so that the entrance/exit of the mobility vehicle or the traveling direction of the mobility vehicle is aligned with the opening portion.

When the mobility vehicle enters the base plate, a sensor may recognize a position of the contact portion, and the connection portion may move toward the contact portion.

In a state in which the mobility vehicle is aligned by the base plate, the multifunctional plate may be matched with an external peripheral shape of the mobility vehicle or formed to be greater than the external peripheral shape.

A first lighting portion may be provided on an upper surface of the multifunctional plate, and the first lighting portion may be turned on and implement a lighting function when the multifunctional plate moves to a lower side of the accommodation space.

A second lighting portion may be provided on a lower surface of the multifunctional plate, and the second lighting portion may be turned on and implement a lighting function when the multifunctional plate moves to an upper side of the accommodation space.

A third lighting portion may be provided at an upper side of the accommodation space of the elevator, and the third lighting portion may be turned on and implement a lighting function when the multifunctional plate moves to a lower side of the accommodation space.

A vehicle washing portion configured to wash the mobility vehicle may be provided on the multifunctional plate, and the vehicle washing portion may provide a vehicle washing function while operating when a door of the elevator and the entrance/exit of the mobility vehicle are closed.

The multifunctional plate moves upward or downward together with the vehicle washing portion when the vehicle washing function is provided.

A first sterilization portion configured to sterilize the accommodation space of the elevator may be provided on the multifunctional plate and provide a sterilization function.

The first sterilization portion may operate after the passenger completely gets out of the accommodation space of the elevator.

A second sterilization portion may be provided at an upper side of the accommodation space of the elevator, and the second sterilization portion may sterilize the multifunctional plate.

The multifunctional plate may further include a cleaning portion configured to draw foreign substances existing on a bottom portion of the accommodation space and the base plate when the multifunctional plate moves downward, and the cleaning portion may provide a cleaning function.

At least one sensing portion may be provided on the multifunctional plate, and the sensing portion may measure a distance between the multifunctional plate and the mobility vehicle and identify a position at which the multifunctional plate and the mobility vehicle do not interfere with each other.

The base plate may align a rotation position based on information inputted through the sensing portion so that the mobility vehicle and the multifunctional plate do not interfere with each other.

Meanwhile, the present disclosure provides a building including an elevating apparatus for a mobility vehicle, the building including: a structure configured as a multi-storied structure and including a passageway configured to connect floors, and at least one opening/closing portion; an elevator configured to be movable upward or downward along the passageway of the structure and including an accommodation space configured to accommodate a mobility vehicle, and at least one opening portion; a base plate provided in the elevator, configured to be rotatable on a bottom surface of the accommodation space, and configured to align the entrance/exit of the mobility vehicle and the opening portion while rotating in a state in which the mobility vehicle is loaded; and a multifunctional plate provided in the elevator and configured to move in an upward/downward direction in the accommodation space, the multifunctional plate being configured to cover the base plate between the entrance/exit of the mobility vehicle and the opening portion of the elevator while moving downward to provide a foothold function to a passenger.

When the elevator moves to a floor level at which the passenger gets out of the mobility vehicle or the mobility vehicle exits, the opening/closing portion of the structure may be opened after the multifunctional plate moves to a lower side of the elevator in a state in which the entrance/exit of the mobility vehicle and the opening portion are aligned by the base plate.

According to the elevating apparatus for a mobility vehicle and the building including the same structured as described above, the vehicle washing function is provided in the elevator to wash the mobility vehicle for the time period for which the mobility vehicle is moved by the elevator, reducing the time and costs required to wash the mobility vehicle.

Furthermore, the foothold function is provided to the passenger who gets in or out of the mobility vehicle, allowing the passenger to conveniently get in or out of the mobility vehicle.

Furthermore, a clean state in the elevator is maintained, a sense of luxury is provided to the passenger, who utilizes the elevator, by implementing various lighting functions in the elevator, and the elevator usage satisfaction is ensured by guiding the boarding or alighting direction for the passenger or the exiting or entering direction for the mobility vehicle by providing lighting.

The effects obtained by the present disclosure are not limited to the aforementioned effects, and other effects, which are not mentioned above, will be clearly understood by those skilled in the art from the following description.

### BRIEF DESCRIPTION OF THE DRAWINGS

FIG. 1 is a view exemplarily illustrating an elevating apparatus for a mobility vehicle and a building including the same according to an exemplary embodiment of the present disclosure.

FIG. 2 is a view exemplarily illustrating the elevating apparatus for a mobility vehicle illustrated in FIG. 1.

FIG. 3 is a view exemplarily illustrating an elevator of the elevating apparatus for a mobility vehicle illustrated in FIG. 1 and a mobility vehicle.

FIG. 4 is a view exemplarily illustrating a downward movement of a multifunctional plate in the elevator of the elevating apparatus for a mobility vehicle illustrated in FIG. 1.

FIG. 5 is a view exemplarily illustrating a state in which the multifunctional plate is moved downwards in the elevator of the elevating apparatus for a mobility vehicle illustrated in FIG. 1.

FIG. 6 is a view exemplarily illustrating a state made before the mobility vehicle enters a base plate in the elevating apparatus for a mobility vehicle according to an exemplary embodiment of the present disclosure.

FIG. 7 is a view exemplarily illustrating a state made after the mobility vehicle enters the base plate in the elevating apparatus for a mobility vehicle according to an exemplary embodiment of the present disclosure.

FIG. 8 is a view exemplarily illustrating a state in which a position of the mobility vehicle is aligned by the base plate in the elevating apparatus for a mobility vehicle according to an exemplary embodiment of the present disclosure.

FIG. 9 is a view exemplarily illustrating components including a foothold function, a lighting function, and a sterilization function of the elevating apparatus for a mobility vehicle according to an exemplary embodiment of the present disclosure.

FIG. 10 is a view exemplarily illustrating a state in which the multifunctional plate is moved downwards in the elevating apparatus for a mobility vehicle according to an exemplary embodiment of the present disclosure.

FIG. 11 is a view exemplarily illustrating a vehicle washing function of the elevating apparatus for a mobility vehicle according to an exemplary embodiment of the present disclosure.

### DETAILED DESCRIPTION OF THE DISCLOSURE

In the description of the embodiments disclosed in the present specification, the specific descriptions of publicly known related technologies will be omitted when it is determined that the specific descriptions may obscure the subject matter of the embodiments disclosed in the present specification. In addition, it should be interpreted that the accompanying drawings are provided only to allow those skilled in the art to easily understand the embodiments disclosed in the present specification, and the technical spirit disclosed in the present specification is not limited by the accompanying drawings, and includes all alterations, equivalents, and alternatives that are included in the spirit and the technical scope of the present disclosure.

The terms including ordinal numbers such as “first,” “second,” and the like may be used to describe various constituent elements, but the constituent elements are not limited by the terms. These terms are used only to distinguish one constituent element from another constituent element.

Singular expressions include plural expressions unless clearly referred to as different meanings in the context.

In the present specification, the terms “comprises,” “comprising,” “includes,” “including,” “containing,” “has,” “having” or other variations thereof are inclusive and therefore specify the presence of stated features, integers, steps, operations, elements, components, and/or combinations thereof, but do not preclude the presence or addition of one or more other features, integers, steps, operations, elements, components, and/or combinations thereof.

The suffixes “module”, “unit”, “part”, and “portion” used to describe constituent elements in the following description are used together or interchangeably to facilitate the description, but the suffixes themselves do not have distinguishable meanings or functions.

When one constituent element is described as being “coupled” or “connected” to another constituent element, it should be understood that one constituent element can be coupled or connected directly to another constituent element, and an intervening constituent element can also be present between the constituent elements. When one constituent element is described as being “coupled directly to” or “connected directly to” another constituent element, it should be understood that no intervening constituent element is present between the constituent elements.

In addition, the term “control unit” or “unit” included in the names is merely a term widely used to name a control device (controller or control unit) for controlling a particular vehicle function but does not mean a generic function unit.

A controller may include a communication device configured to communicate with another control unit or a sensor to control a corresponding function, a memory configured to store an operating system, a logic instruction, and input/output information, and one or more processors configured to perform determination, computation, decision, or the like required to control the corresponding function.

In any configuration among the configurations disclosed in the present specification, any number of components or various components may be included in the present disclosure disclosed in the present specification. The components may include any combination of the features disclosed in the present specification and be arranged in any configuration among various configurations disclosed in the present specification. The concepts related to the use and operations of the components of the present disclosure, as well as the structure and arrangement of the components of the present disclosure, may be applied not only to particular embodiments discussed in the present specification but also to any number of embodiments in any combination. The exemplary embodiments including various features of various arrangements will be described below with reference to the drawings.

Hereinafter, various embodiments disclosed in the present specification will be described in detail with reference to the accompanying drawings. The same or similar constituent elements are assigned with the same reference numerals regardless of reference numerals, and the repetitive description thereof will be omitted.

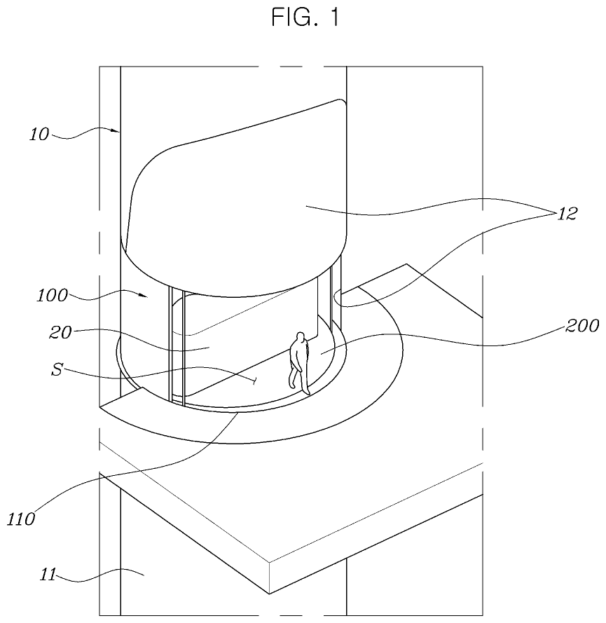

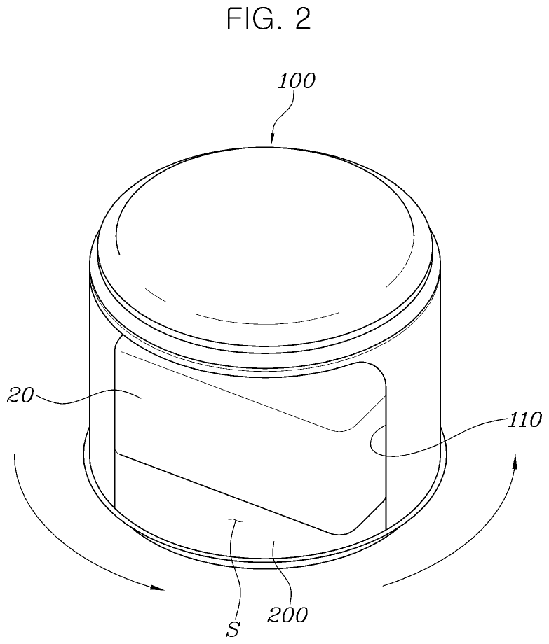

FIG. 1 is a view exemplarily illustrating an elevating apparatus for a mobility vehicle according to an exemplary embodiment of the present disclosure, the elevating apparatus including a structure and an elevator 100, and FIG. 2 is a view exemplarily illustrating the elevator 100 and a mobility vehicle 20.

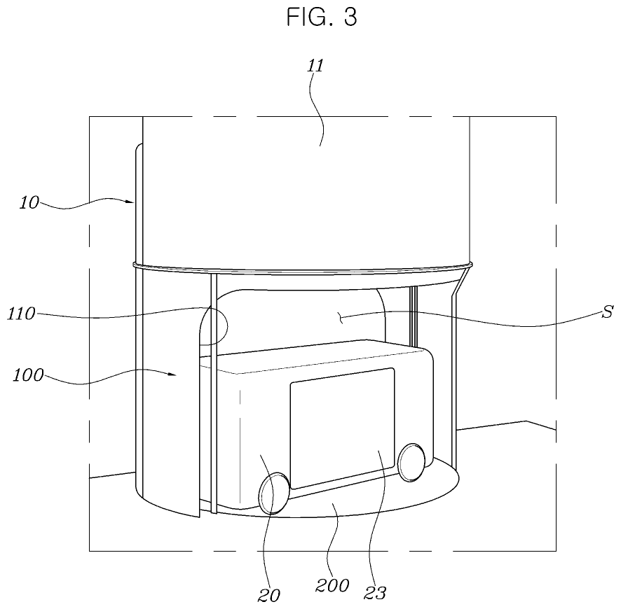

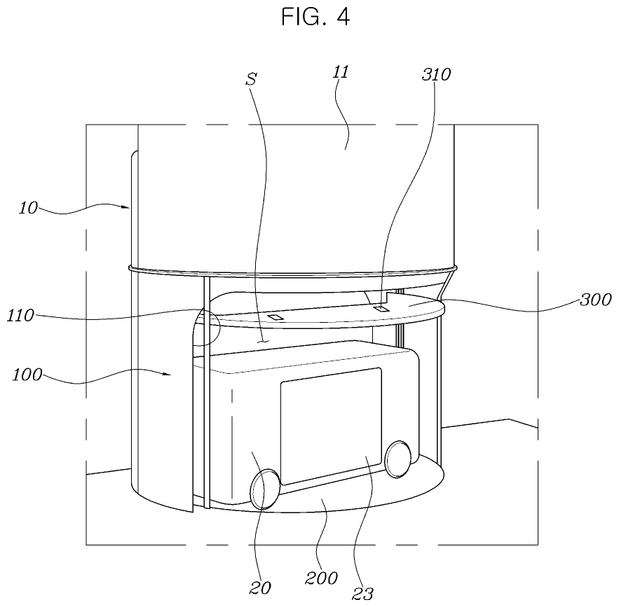

Furthermore, FIGS. 3 to 5 are views exemplarily illustrating states in which the mobility vehicle 20 enters an accommodation space S in the elevator 100 and is loaded onto a base plate 200, and a multifunctional plate 300 moves upward or downward.

As illustrated in FIGS. 1 to 5, the elevating apparatus for a mobility vehicle according to an exemplary embodiment of the present disclosure includes the elevator 100 disposed in a structure 10, configured to be movable upward or downward, including the accommodation space S configured to accommodate the mobility vehicle 20, and including at least one opening portion 110, the base plate 200 disposed in the elevator 100, configured to be rotatable on a bottom surface of the accommodation space S, and configured to align an entrance/exit 21 of the mobility vehicle 20 or a traveling direction of the mobility vehicle 20 with the opening portion 110 while rotating in the state in which the mobility vehicle 20 is loaded, and the multifunctional plate 300 disposed in the elevator 100 and configured to move in an upward/downward direction in the accommodation space S, the multifunctional plate 300 being configured to cover the base plate 200 between the entrance/exit 21 of the mobility vehicle 20 and the opening portion 110 of the elevator 100 while moving downward to provide the foothold function to the passenger.

In an exemplary embodiment of the present disclosure, the structure 10 may be a building, a parking tower, or the like and configured as a multi-storied structure.

The structure 10 includes the elevator 100 that moves upward or downward to a plurality of floor levels. The structure in which the elevator 100 moves upward or downward in the structure 10 is an apparent configuration generally applied to a typical elevator structure and deviates from a key point of the present disclosure. Therefore, a specific description thereof will be omitted.

The accommodation space S is disposed inside the elevator 100, and the accommodation space S is formed to accommodate the mobility vehicle 20. In addition, at least one opening portion 110 may be formed in the elevator 100 so that the mobility vehicle 20 may move to the inside or outside of the accommodation space S through the opening portion 110. In addition, the elevator 100 may include a door configured to open or close the opening portion 110, and the opening portion 110 may be in an opened state without the door.

Meanwhile, the base plate 200 and the multifunctional plate 300 are disposed in the accommodation space S of the elevator 100.

The base plate 200 is disposed in the elevator 100 and configured to be rotatable on the bottom surface of the accommodation space S. When the mobility vehicle 20 enters the accommodation space S and is loaded onto the base plate 200, the base plate 200 may rotate to change a position of the mobility vehicle 20.

The base plate 200 may be rotatably disposed in the elevator 100 by various means such as at least any one of a motor, an actuator, and a gear connection structure. In addition, various ways for rotating objects may be applied. In addition, the base plate 200 may be formed to be wider than the mobility vehicle 20 so that the mobility vehicle 20 may be positioned inside the base plate 200 when the mobility vehicle 20 is mounted. As described above, the base plate 200 may be provided in the form of a circular plate and formed to be larger in area than the mobility vehicle 20 when viewed from above to below so that the mobility vehicle 20 may be accommodated inside the base plate 200.

As described above, the base plate 200 is configured to rotate in the accommodation space S of the elevator 100 so that the entrance/exit 21 of the mobility vehicle 20 or the traveling direction of the mobility vehicle 20 may be aligned with the opening portion 110 in accordance with a rotation position.

That is, in case that the mobility vehicle 20 is unloaded from the elevator 100 after the mobility vehicle 20 enters the accommodation space S of the elevator 100, the traveling direction of the mobility vehicle 20 and the opening portion 110 of the elevator 100 are aligned by the rotation of the base plate 200 so that the mobility vehicle 20 may be unloaded from the elevator 100 as the mobility vehicle 20 merely travels straight.

Furthermore, in case that only a passenger gets out of the elevator 100 after the mobility vehicle 20 enters the accommodation space S of the elevator 100, the entrance/exit 21 of the mobility vehicle 20 and the opening portion 110 of the elevator 100 are aligned by the rotation of the base plate 200, the passenger, who gets out of the mobility vehicle 20, may easily exit to the opening portion 110 of the elevator 100.

The rotation position of the base plate 200 may be adjusted under the control of a controller C disposed in the structure 10 or the elevator 100. The controller C may communicate with the mobility vehicle 20, identify whether the mobility vehicle 20 exits or the passenger gets in or out of the mobility vehicle 20, and control the rotation position of the base plate 200.

The multifunctional plate 300 is disposed in the elevator 100 and configured to move in the upward/downward direction in the accommodation space S. With reference to FIGS. 3 to 5, the multifunctional plate 300 may move upward or downward in the accommodation space S by a rail structure. That is, a rail configured to allow the multifunctional plate 300 to move upward or downward may be disposed in the accommodation space S of the elevator 100, and the multifunctional plate 300 may be coupled to the rail and move upward or downward. As a rail connection structure of the multifunctional plate 300, various one or more structures, such as a rack-and-pinion structure, a chain connection structure, and a gear connection structure, may be applied. Furthermore, connection structures for moving objects in rectilinear directions may be selectively applied.

As described above, the multifunctional plate 300 moves in the upward/downward direction in the accommodation space S of the elevator 100. In case that the multifunctional plate 300 moves upward, the multifunctional plate 300 does not interfere with the mobility vehicle 20. In case that the multifunctional plate 300 moves downward, the multifunctional plate 300 provides the foothold function to the passenger by covering the base plate 200 between the entrance/exit 21 of the mobility vehicle 20 and the opening portion 110 of the elevator 100.

Therefore, when the passenger gets in or out of the mobility vehicle 20, a gap between the entrance/exit 21 of the mobility vehicle 20 and the structure 10 is covered by the base plate 200 so that the passenger may get in or out of the mobility vehicle 20 without discomfort caused by a height deviation.

A vertical position of the multifunctional plate 300 may be adjusted under the control of the controller C disposed in the structure 10 or the elevator 100. The controller C may communicate with the mobility vehicle 20, identify whether the mobility vehicle 20 exits or the passenger gets in or out of the mobility vehicle 20, and control the vertical position of the multifunctional plate 300.

The above-mentioned present disclosure will be described below specifically.

In an exemplary embodiment of the present disclosure, when the mobility vehicle 20 enters the accommodation space S of the elevator 100, the base plate 200 may rotate to align the position of the mobility vehicle 20.

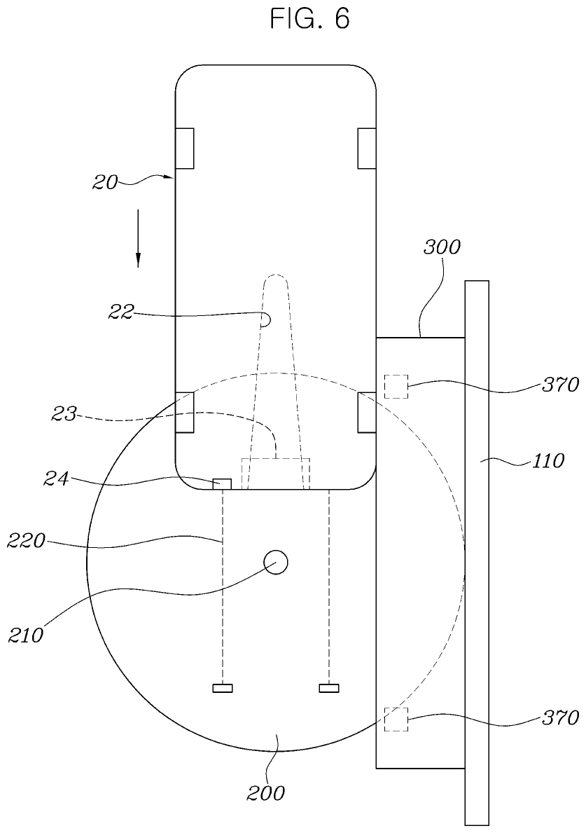

FIGS. 6 to 8 illustrate that the mobility vehicle 20 enters the base plate 200, and the position of the mobility vehicle 20 is aligned.

A contact portion 210 is disposed at a center portion of the base plate 200, and a connection portion 22 configured to come into contact with the contact portion 210 is disposed on a lower portion of the mobility vehicle 20 so that when the mobility vehicle 20 enters the base plate 200, the connection portion 22 is guided while coming into contact with the contact portion 210 so that the position of the mobility vehicle 20 may be aligned with the center portion of the base plate 200.

The contact portion 210 may protrude upwards from a surface of the base plate 200. In another embodiment, the contact portion 210 may be configured to be selectively extended or retracted. For example, the contact portion 210 may be configured to selectively protrude from the base plate 200 by an actuator. The contact portion 210 may be retracted in case that the mobility vehicle does not enter the accommodation space S of the elevator 100. In case that the mobility vehicle 20 enters the accommodation space S, the contact portion 210 may be extended from the base plate 200 and come into contact with the connection portion 22 of the mobility vehicle 20.

The contact portion 210 is disposed at the center portion of the base plate 200 so that in case that the connection portion 22 of the mobility vehicle 20 comes into contact with the contact portion 210 and the movement of the mobility vehicle 20 is guided, the mobility vehicle 20 may move to the center portion of the base plate 200 to suit the position of the contact portion 210 of the base plate 200.

The connection portion 22 is formed on the lower portion of the mobility vehicle 20. When the mobility vehicle 20 enters the accommodation space S of the elevator 100 and is loaded onto the base plate 200, the connection portion 22 comes into contact with the contact portion 210 so that a movement direction of the mobility vehicle 20 may be guided.

In the instant case, the connection portion 22 may be configured as a slit extending in a traveling direction in the lower portion of the mobility vehicle 20. When the mobility vehicle 20 enters the base plate 200, the contact portion 210 is inserted into the connection portion 22, and the movement of the connection portion 22 is guided so that the position of the mobility vehicle 20 may be fixed after the mobility vehicle 20 moves to the center portion of the base plate 200 at which the contact portion 210 is formed.

The connection portion 22 extends so that a width thereof gradually decreases from a lower rim of the mobility vehicle 20 toward the mobility vehicle 20. Therefore, when the mobility vehicle 20 enters the base plate 200, the contact portion 210 and the connection portion 22 may guide the mobility vehicle 20 so that the mobility vehicle 20 is positioned at the center portion of the base plate 200.

The connection portion 22 may be formed on the lower portion of the mobility vehicle 20 and extend from the rim toward the center portion of the mobility vehicle 20. For example, the connection portion 22 may extend from a front side of the mobility vehicle 20 based on the traveling direction toward a center portion of the mobility vehicle 20 and be formed in a slit shape so that the contact portion 210 may be inserted into the connection portion 22. An inlet of the connection portion 22 is formed to be wider than the contact portion 210 so that the contact portion 210 may be inserted into the connection portion 22 when the mobility vehicle 20 enters the base plate 200. Furthermore, the connection portion 22 is formed so that the width thereof gradually decreases toward the center. As the mobility vehicle 20 enters the base plate 200, the contact portion 210 comes into contact with the inside of the connection portion 22 so that the movement of the mobility vehicle 20 is guided. Finally, the contact portion 210 comes into contact with and is fitted with a distal end portion of the connection portion 22 so that the movement of the mobility vehicle 20 may be restricted.

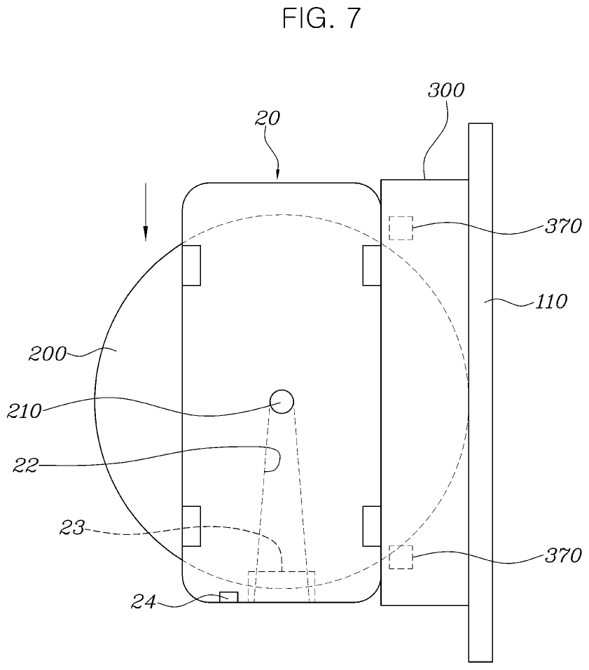

As may be seen in FIGS. 6 to 7, when the mobility vehicle 20 enters the base plate 200, a movement position of the connection portion 22 is guided by the contact portion 210. As the contact portion 210 moves to the distal end portion of the connection portion 22, the position of the mobility vehicle 20 moves to the center portion of the base plate 200 so that the position of the mobility vehicle 20 may be aligned with the center portion of the base plate 200 when the mobility vehicle 20 enters the accommodation space S of the elevator 100.

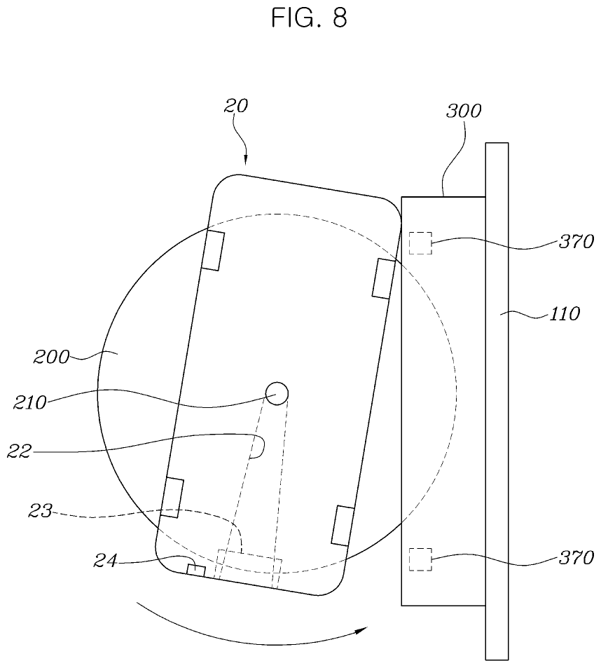

Furthermore, as may be seen in FIG. 8, the entrance/exit 21 of the mobility vehicle 20 or the traveling direction of the mobility vehicle 20 may be aligned with the opening portion 110 by the rotation of the base plate 200. That is, the position of the mobility vehicle 20 is aligned in conjunction with the rotation of the base plate 200 so that the entrance/exit 21 of the mobility vehicle 20 may be matched with the opening portion 110, or the traveling direction of the mobility vehicle 20 may be matched with the opening portion 110.

Therefore, the mobility vehicle 20 may move to the outside from the accommodation space S merely by traveling straight when the mobility vehicle 20 exits the accommodation space S of the elevator 100. When the passenger gets in or out of the mobility vehicle 20, the entrance/exit 21 and the opening portion 110 may be matched with each other so that the passenger may smoothly get in or out of the mobility vehicle 20.

Meanwhile, a door portion 23 may be disposed in the inlet of the connection portion 22 of the mobility vehicle 20. The door portion 23 may be opened in case that the mobility vehicle 20 enters the base plate 200.

The door portion 23 is openably and closably disposed in the inlet of the connection portion 22. At ordinary times, the door portion is in a closed state so that an obstacle other than the contact portion 210 is prevented from entering the connection portion 22 of the mobility vehicle 20. The door portion 23 is opened when the mobility vehicle 20 enters the base plate 200, allowing the contact portion 210 to enter the connection portion 22.

The door portion 23 may be selectively opened or closed upon the passenger's authorization. Alternatively, when the mobility vehicle 20 enters the accommodation space S of the elevator 100, the door portion 23 may be opened or closed while communicating with the controller C disposed in the structure 10 or the elevator 100.

The opening/closing structure of the door portion 23 may be configured to be opened or closed in a rotating or sliding manner in the inlet of the connection portion 22, and opening and closing positions may be switched under the control of a motor or an actuator by the controller C.

Meanwhile, the mobility vehicle 20 may include a sensor 24. At least any one or more of a camera, an infrared sensor, a laser sensor, and a Light Detection and Ranging (LiDAR) may be applied as the sensor 24. Furthermore, various means for detecting an object present in the traveling direction of the mobility vehicle 20 may be selectively applied.

Therefore, when the mobility vehicle 20 enters the base plate 200, the sensor 24 may recognize the position of the contact portion 210, and the connection portion 22 may move toward the contact portion 210 as the mobility vehicle 20 enters the base plate 200 to align with the contact portion 210.

That is, when the mobility vehicle 20 enters the accommodation space S of the elevator 100, the sensor 24 may identify the base plate 200 and recognize the position of the contact portion 210 formed on the base plate 200, and the connection portion 22 may move toward the contact portion 210. In the instant case, when the mobility vehicle 20 moves in a direction in which the mobility vehicle 20 is loaded onto the base plate 200, the mobility vehicle 20 may automatically travel so that the connection portion 22 is directed toward the contact portion 210.

Furthermore, a guide line 220 for guiding an entering direction of the mobility vehicle 20 is further formed on the base plate 200 so that the sensor 24 of the mobility vehicle 20 may detect the guide line 220, the mobility vehicle 20 may move along the guide line 220, and the movement of the mobility vehicle 20 may be guided toward the center portion of the base plate 200.

Therefore, when the mobility vehicle 20 enters the accommodation space S of the elevator 100, the mobility vehicle 20 travels so that the connection portion 22 comes into contact with the contact portion 210 and the movement position is automatically aligned toward the center portion of the base plate 200 even if the passenger does not directly manipulate the mobility vehicle 20. Therefore, the mobility vehicle 20 is easily positioned at an exact position on the base plate 200.

As described above, in the present disclosure, the position of the mobility vehicle 20 may be aligned with the center portion of the base plate 200 by the connection portion 22 and the contact portion 210, and the convenience related to the situation in which the mobility vehicle 20 exits or the passenger gets out of the mobility vehicle 20 may be ensured in accordance with the position at which the base plate 200 rotates.

That is, when the mobility vehicle 20 enters the accommodation space S of the elevator 100, the mobility vehicle 20 is guided to the center portion of the base plate 200. In case that the mobility vehicle 20 exits the elevator 100, the base plate 200 rotates so that the traveling direction of the mobility vehicle 20 and the opening portion 110 of the elevator 100 are aligned, the mobility vehicle 20 may exit the elevator 100 merely by traveling straight.

Furthermore, in case that only the passenger gets out of the elevator 100, the base plate 200 rotates so that the entrance/exit 21 of the mobility vehicle 20 and the opening portion 110 of the elevator 100 are aligned so that the passenger, who gets out of the mobility vehicle 20, may exit to the opening portion 110 of the elevator 100.

Meanwhile, in the present disclosure, the multifunctional plate 300 may implement a lighting function and a vehicle washing function as well as the foothold function in the accommodation space S of the elevator 100.

In the state in which the mobility vehicle 20 is aligned by the base plate 200, the multifunctional plate 300 may be matched with an external peripheral shape of the mobility vehicle 20 or formed to be greater than the external peripheral shape.

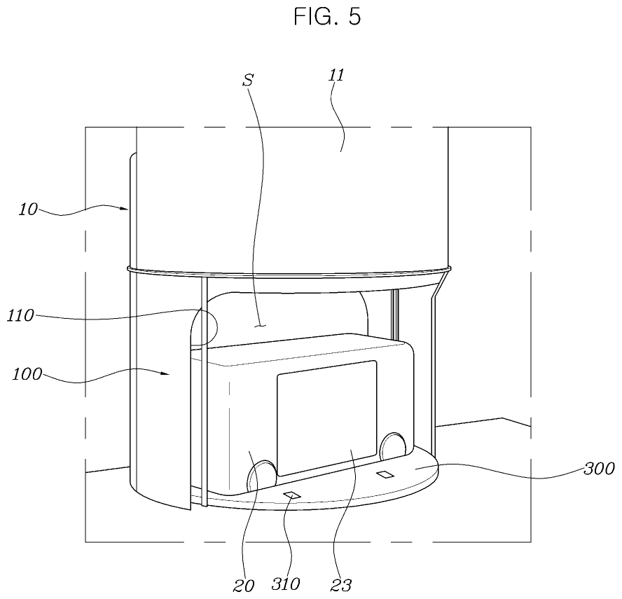

With reference to FIG. 5, the multifunctional plate 300 may be disposed between the mobility vehicle 20 and the opening portion 110 of the elevator 100 in the state in which the position of the mobility vehicle 20 is aligned by the base plate 200.

As described above, the multifunctional plate 300 is formed to be matched with the external peripheral shape of the mobility vehicle 20, a gap between the mobility vehicle 20 and the opening portion 110 may be minimized when the multifunctional plate 300 is positioned between the entrance/exit 21 of the mobility vehicle 20 and the opening portion 110 of the elevator 100.

As described above, when the foothold function is implemented, the multifunctional plate 300 may cover a portion between the mobility vehicle 20 and the opening portion 110, ensuring stability and convenience related to the situation in which the passenger gets in or out of the mobility vehicle 20.

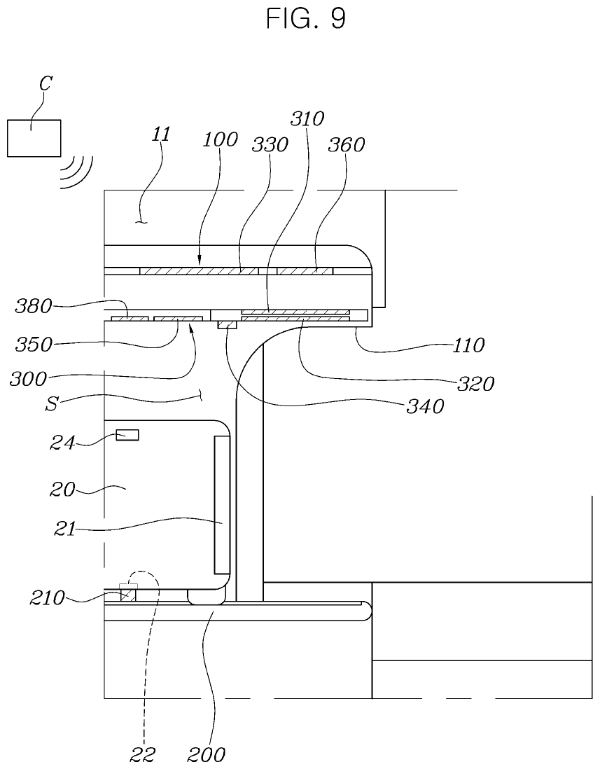

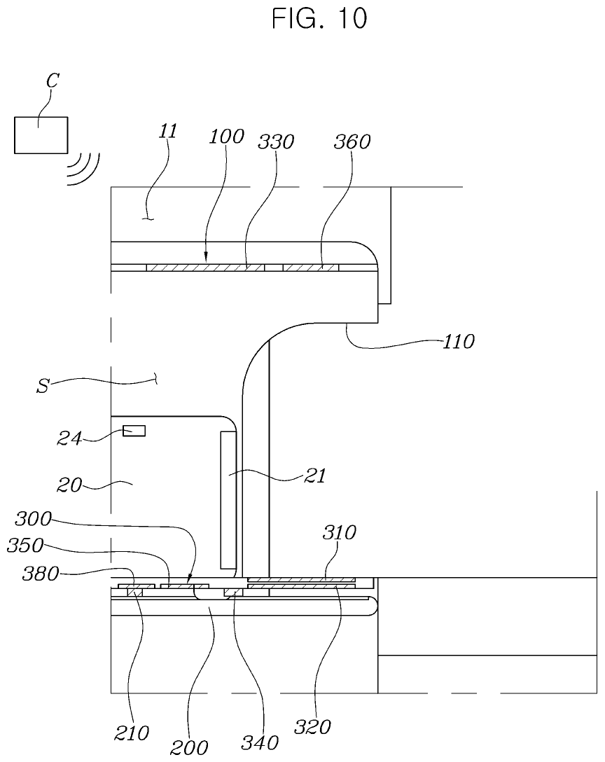

Meanwhile, FIG. 9 is a view exemplarily illustrating a state in which the multifunctional plate 300 is moved to an upper side of the accommodation space S, and FIG. 10 is a view exemplarily illustrating a state in which the multifunctional plate 300 is moved to a lower side of the accommodation space S.

A first lighting portion 310 may be disposed on an upper surface of the multifunctional plate 300, and the first lighting portion 310 may be turned on and implement the lighting function when the multifunctional plate 300 moves to the lower side of the accommodation space S.

The first lighting portion 310 may be configured by an LED. The first lighting portion 310 may be disposed on the upper surface of the multifunctional plate 300 and emit light upwards. In case that the multifunctional plate 300 moves downward in the accommodation space S, the first lighting portion 310 may be exposed from the upper surface of the multifunctional plate 300, and the first lighting portion 310 may be turned on so that various lighting functions may be implemented by use of light.

For example, the first lighting portion 310 may be configured to generate an image of light, which connects the entrance/exit 21 of the mobility vehicle 20 and the opening portion 110 of the elevator 100, on the upper surface of the multifunctional plate 300, guiding a boarding or alighting direction for the passenger. Furthermore, the first lighting portion 310 may be turned on and generate an image of light in the accommodation space S of the elevator 100 in the state in which the multifunctional plate 300 is moved downward, and the image of light may also be utilized as a design element.

Meanwhile, a second lighting portion 320 may be disposed on a lower surface of the multifunctional plate 300, and the second lighting portion 320 may be turned on and implement the lighting function when the multifunctional plate 300 moves to the upper side of the accommodation space S.

The second lighting portion 320 may be configured by an LED. The second lighting portion 320 may be disposed on the lower surface of the multifunctional plate 300 and emit light downward. In case that the multifunctional plate 300 moves upward in the accommodation space S, the second lighting portion 320 may be exposed from the lower surface of the multifunctional plate 300, and the second lighting portion 320 may be turned on so that various lighting functions may be implemented by use of light.

For example, when the mobility vehicle 20 exits the accommodation space S of the elevator 100, the second lighting portion 320 may emit light toward the base plate 200 and guide an exiting direction for the mobility vehicle 20. Furthermore, the second lighting portion 320 may be turned on and generate an image of light in the accommodation space S of the elevator 100, and the image of light may also be utilized as a design element.

Meanwhile, the elevator 100 may include a third lighting portion 330 disposed at the upper side of the accommodation space S. When the multifunctional plate 300 moves to the lower side of the accommodation space S, the third lighting portion 330 may be turned on and implement the lighting function.

The third lighting portion 330 may be configured as an LED. The third lighting portion 330 may be disposed in the elevator 100, disposed above the multifunctional plate 300, and configured to emit light toward the multifunctional plate 300. In case that the multifunctional plate 300 moves downward in the accommodation space S, the third lighting portion 330 may emit light to the upper surface of the multifunctional plate 300, and the third lighting portion 330 may be turned on so that various lighting functions may be implemented on the multifunctional plate 300 by use of light.

For example, when the multifunctional plate 300 moves downward, the third lighting portion 330 may emit light to the upper surface of the multifunctional plate 300 to guide the passenger who gets in or out of the mobility vehicle, or the third lighting portion 330 may be configured to generate an image of light so that the image of light may be utilized as a design element.

Furthermore, the third lighting portion 330 may be turned on even in the state in which the multifunctional plate 300 is moved upwards. When the light is emitted in the state in which the multifunctional plate 300 is moved upward, a mood lighting effect may be implemented by reflecting light.

As described above, in the present disclosure, the first, second, and third lighting portions 310, 320, and 330 generate light in the accommodation space S of the elevator 100, which may improve the convenience for the passenger and a sense of luxury in the elevator 100.

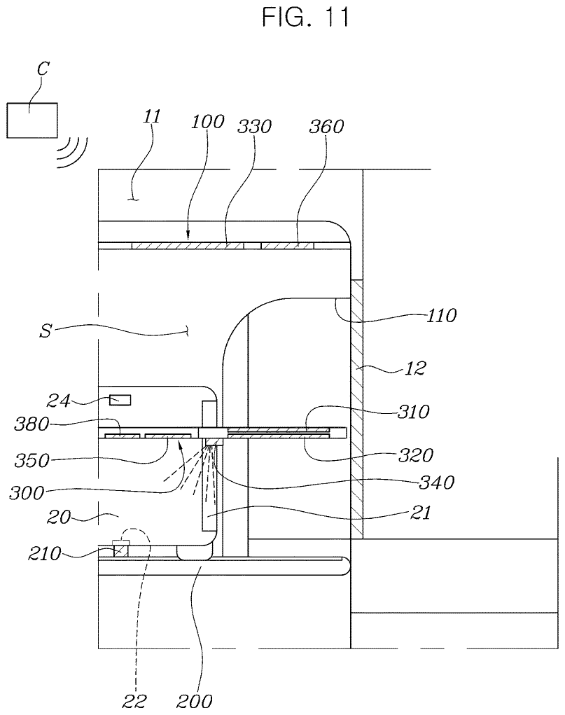

FIG. 11 is a view exemplarily illustrating a state in which the multifunctional plate 300 moves, and a vehicle washing portion 340 operates.

The vehicle washing portion 340 configured to wash the mobility vehicle 20 may be disposed on the multifunctional plate 300. The vehicle washing portion 340 may provide the vehicle washing function by operating when a door of the elevator 100 or the entrance/exit 21 of the mobility vehicle 20 is closed.

Furthermore, when the vehicle washing function is provided, the multifunctional plate 300 moves upward or downward together with the vehicle washing portion 340.

In an exemplary embodiment of the present disclosure, the vehicle washing portion 340 may be disposed on the multifunctional plate 300 and wash the mobility vehicle by use of a brush that rotates while moving together with the multifunctional plate 300, wash the mobility vehicle by spraying high-pressure water without using a brush, or wash the mobility vehicle by use of a device for spraying high-pressure air.

For example, in case that the vehicle washing portion 340 includes the brush, the brush may be disposed on a portion of the multifunctional plate 300 directed toward the mobility vehicle 20. When the multifunctional plate 300 moves upward or downward, the brush may come into contact with an external periphery of the mobility vehicle 20 and wash the mobility vehicle 20.

Furthermore, in case that the vehicle washing portion 340 is configured to spray water, a water storage tank and a pressure pump configured for spraying water may be disposed in the elevator 100, a hose connected to the water storage tank and the pressure pump may extend from the multifunctional plate 300, and a spraying position of the hose may be disposed to be directed toward the mobility vehicle 20. In the instant case, the water storage tank of the elevator 100 may be supplied with water from the structure 10.

Furthermore, in case that the vehicle washing portion 340 is configured to spray air, an air pump may be disposed in the elevator 100.

At least any one of the above-mentioned embodiments may be applied to the vehicle washing portion 340, or the above-mentioned embodiments may be complexly applied to the vehicle washing portion 340. In addition to the above-mentioned embodiments related to the vehicle washing function, various means configured for washing the mobility vehicle 20 may be applied. Furthermore, the above-mentioned embodiments exemplarily propose various embodiments of the vehicle washing portion 340 and are apparent configurations to which a typical vehicle washing technology is applied, and the above-mentioned embodiments deviate from the key point of the present disclosure. Therefore, a specific description thereof will be omitted.

As described above, in the present disclosure, the vehicle washing portion 340 disposed on the multifunctional plate 300 may wash the mobility vehicle 20. Because the multifunctional plate 300 moves upward or downward in conjunction with the operation of the vehicle washing portion 340, the efficiency in washing the mobility vehicle 20 may be improved.

Meanwhile, a first sterilization portion 350 configured to sterilize the accommodation space S of the elevator 100 is disposed on the multifunctional plate 300 and provides a sterilization function.

The first sterilization portion 350 may be configured to sterilize an internal space of the elevator 100 by emitting ultraviolet rays.

For example, the first sterilization portion 350 may be an ultraviolet sterilization lamp. The first sterilization portion 350 may be configured to emit ultraviolet rays with a single wavelength by singly using any one of an UV-A wavelength LED, an UV-B wavelength LED, and an UV-C wavelength LED or emit ultraviolet rays with a mixed wavelength made by use of at least two of the UV-A wavelength LED, the UV-B wavelength LED, and the UV-C wavelength LED.

The first sterilization portion 350 may operate after the passenger completely gets out of the accommodation space S of the elevator 100.

That is, the first sterilization portion 350 operates in case that the detection sensor 24 disposed in the accommodation space S of the elevator 100 identifies that there is no person or animal present in the accommodation space S or in case that the first sterilization portion 350 communicates with the mobility vehicle 20, and various types of sensors, which are disposed in the mobility vehicle 20, identify that there is no person or animal present in the accommodation space S. Therefore, it is possible to prevent damage to a person or animal caused by the emission of ultraviolet rays.

Furthermore, whether the mobility vehicle 20 exits the accommodation space S of the elevator 100 as well as whether the passenger gets out of the accommodation space S of the elevator 100 is further identified, and the first sterilization portion 350 may operate in case that the mobility vehicle 20 is not present in the accommodation space S. Therefore, the efficiency in sterilizing the accommodation space S by the first sterilization portion 350 may be ensured, which may improve the satisfaction related to the use of the elevator 100 later.

Meanwhile, the elevator 100 may include a second sterilization portion 360 disposed at the upper side of the accommodation space S, and the second sterilization portion 360 may sterilize the multifunctional plate 300.

The second sterilization portion 360 may be disposed in the elevator 100, positioned above the multifunctional plate 300, and configured to emit ultraviolet rays toward the multifunctional plate 300.

For example, the second sterilization portion 360 may be an ultraviolet sterilization lamp. The second sterilization portion 360 may be configured to emit ultraviolet rays with a single wavelength by singly using any one of an UV-A wavelength LED, an UV-B wavelength LED, and an UV-C wavelength LED or emit ultraviolet rays with a mixed wavelength made by use of at least two of the UV-A wavelength LED, the UV-B wavelength LED, and the UV-C wavelength LED.

The second sterilization portion 360 may operate when the multifunctional plate 300 moves upward, and the second sterilization portion 360 may emit ultraviolet rays to the multifunctional plate 300 moved upward, suppressing the growth of bacteria on the multifunctional plate 300. That is, the multifunctional plate 300 may move downward to serve as a foothold for the passenger and perform the vehicle washing function so that contamination and bacteria growth are suppressed by the second sterilization portion 360.

Meanwhile, a cleaning portion 380 may be further disposed on the multifunctional plate 300 and draw foreign substances existing on a bottom portion of the accommodation space S and the base plate 200 when the multifunctional plate 300 moves downward, providing a cleaning function.

The cleaning portion 380 may include a suction port for drawing dust and foreign substances. The elevator 100 may further include a storage configured to store dust and foreign substances drawn in by the cleaning portion 380, and a suction pump.

Because the cleaning portion 380 is an apparent component for a vacuum cleaner, a detailed description thereof will be omitted. The suction port for drawing dust and foreign substances may be provided on an external periphery or a lower portion of the multifunctional plate 300.

Therefore, when the multifunctional plate 300 moves downward, the cleaning portion 380 may operate to draw dust and foreign substances existing on the base plate 200 and the bottom portion of the accommodation space S and clean the accommodation space S of the elevator 100 so that a comfortable state of the accommodation space S of the elevator 100 may be maintained.

Meanwhile, at least one sensing portion 370 may be disposed on the multifunctional plate 300. The sensing portion 370 measures a distance between the multifunctional plate 300 and the mobility vehicle 20 and identifies a position at which the multifunctional plate 300 and the mobility vehicle 20 do not interfere with each other.

The sensing portion 370 is for detecting a distance between objects. At least one of a laser sensor, a LiDAR, an infrared sensor, and a camera may be applied. Furthermore, various embodiments may be applied to means for detecting distances between objects.

That is, the sensing portion 370 is disposed on the multifunctional plate 300, and the sensing portion 370 measures a distance between the multifunctional plate 300 and the mobility vehicle 20. It is possible to recognize whether the multifunctional plate 300 may move downward based on a spacing distance between the multifunctional plate 300 and the mobility vehicle 20 measured by the sensing portion 370 as described above, and it is possible to identify the position, at which the multifunctional plate 300 and the mobility vehicle 20 do not interfere with each other, and to align the position of the mobility vehicle 20 by rotating the base plate 200.

The sensing portion 370 may be disposed as a plurality of sensing portions 370 disposed on the multifunctional plate 300. The sensing portions 370 may each measure a distance between the multifunctional plate 300 and the mobility vehicle 20, recognizing the current position of the mobility vehicle 20 and identifying whether the multifunctional plate 300 and the mobility vehicle 20 interfere with each other.

Therefore, the base plate 200 may align a rotation position based on information inputted through the sensing portion 370 so that the mobility vehicle 20 and the multifunctional plate 300 do not interfere with each other.

That is, when the mobility vehicle 20 enters the accommodation space S of the elevator 100 and is loaded onto the base plate 200, the sensing portion 370 may measure the distance between the multifunctional plate 300 and the mobility vehicle 20 and identify a degree to which the multifunctional plate 300 and the mobility vehicle 20 interfere with each other. Based on the information obtained by the sensing portion 370, it is possible to recognize the position at which the mobility vehicle 20 and the multifunctional plate 300 do not interfere with each other. Therefore, it is possible to realign the position of the mobility vehicle 20 by rotating the base plate 200 and prevent the multifunctional plate 300 and the mobility vehicle 20 from interfering with each other.

Based on the information obtained by the sensing portion 370, the controller C may derive the position at which the multifunctional plate 300 and the mobility vehicle 20 do not interfere with each other, and the controller C may be configured to determine the rotation position of the base plate 200.

Meanwhile, a building disposed with the elevating apparatus for a mobility vehicle according to the exemplary embodiment of the present disclosure includes the structure 10 configured as a multi-storied structure and including a passageway 11 configured to connect floors, and at least one opening/closing portion 12, the elevator 100 configured to be movable upward or downward along the passageway 11 of the structure 10 and including the accommodation space S configured to accommodate the mobility vehicle 20, and at least one opening portion 110, the base plate 200 disposed in the elevator 100, configured to be rotatable on the bottom surface of the accommodation space S, and configured to align the entrance/exit 21 of the mobility vehicle 20 and the opening portion 110 while rotating in the state in which the mobility vehicle 20 is loaded, and the multifunctional plate 300 disposed in the elevator 100 and configured to move in the upward/downward direction in the accommodation space S, the multifunctional plate 300 being configured to cover the base plate 200 between the entrance/exit 21 of the mobility vehicle 20 and the opening portion 110 of the elevator 100 while moving downward to provide the foothold function to the passenger.

The structure 10 may be a building, a parking tower, or the like and configured as a multi-storied structure.

The structure 10 may include the passageway 11 configured to connect a plurality of floor levels. The elevator 100 may move upward or downward in the passageway 11. The opening/closing portion 12 may be disposed at a portion of the passageway 11 matched with each of the floor levels. The opening/closing portion 12 may be a door that separates a space in the structure 10 and the passageway 11. The opening/closing portion 12 may be selectively opened or closed by being controlled by a controller disposed in the structure 10.

The base plate 200 is disposed in the elevator 100 and configured to be rotatable on the bottom surface of the accommodation space S. When the mobility vehicle 20 enters the accommodation space S and is loaded onto the base plate 200, the base plate 200 may rotate to change the position of the mobility vehicle 20.

The multifunctional plate 300 moves in the upward/downward direction in the accommodation space S of the elevator 100. In case that the multifunctional plate 300 moves upward, the multifunctional plate 300 does not interfere with the mobility vehicle 20. In case that the multifunctional plate 300 moves downward, the multifunctional plate 300 provides the foothold function to the passenger by covering the base plate 200 between the entrance/exit 21 of the mobility vehicle 20 and the opening portion 110 of the elevator 100.

According to an exemplary embodiment of the present disclosure, when the elevator 100 moves to the floor level, at which the passenger intends to get out of the elevator 100, after the mobility vehicle 20 enters the accommodation space S of the elevator 100, the entrance/exit 21 of the mobility vehicle 20 and the opening portion 110 are aligned by the rotation of the base plate 200. After the multifunctional plate 300 is moved to the lower side of the elevator 100, the opening/closing portion 12 of the structure 10 is opened so that the gap between the entrance/exit 21 of the mobility vehicle 20 and the structure 10 is covered by the base plate 200, and the passenger may stably get in or out of the mobility vehicle 20 without discomfort caused by a height deviation.

According to the elevating apparatus for a mobility vehicle and the building including the same structured as described above, the vehicle washing function is provided in the elevator to wash the mobility vehicle for the time period for which the mobility vehicle is moved by the elevator, reducing the time and costs required to wash the mobility vehicle.

Furthermore, the foothold function is provided to the passenger who gets in or out of the mobility vehicle, allowing the passenger to conveniently get in or out of the mobility vehicle.

Furthermore, a clean state in the elevator is maintained, a sense of luxury is provided to the passenger, who utilizes the elevator, by implementing various lighting functions in the elevator, and the elevator usage satisfaction is ensured by guiding the boarding or alighting direction for the passenger or the exiting or entering direction for the mobility vehicle by providing lighting.

While the predetermined embodiments of the present disclosure have been illustrated and described, it will be obvious to those skilled in the art that the present disclosure may be variously modified and changed without departing from the technical spirit of the present disclosure defined in the appended claims.

What is claimed is:

1. An elevating apparatus for a mobility vehicle, the elevating apparatus comprising:
an elevator disposed in a structure, configured to be movable upward or downward in the structure, including an accommodation space configured to accommodate the mobility vehicle therein, and including at least one opening portion;
a base plate disposed in the elevator, configured to be rotatable on a bottom surface of the accommodation space, and configured to align an entrance/exit of the mobility vehicle or a traveling direction of the mobility vehicle with the at least one opening portion while rotating in a state in which the mobility vehicle is loaded on the base plate; and
a multifunctional plate disposed in the elevator and configured to move in an upward/downward direction in the accommodation space, the multifunctional plate being configured to cover the base plate between the entrance/exit of the mobility vehicle and the at least one opening portion of the elevator while moving downward to provide a foothold function to a passenger.

2. The elevating apparatus of claim 1, further comprising:
a contact portion disposed at a center portion of the base plate, wherein the contact portion is configured to contact with a connection portion disposed in a lower portion of the mobility vehicle so that the connection portion of the mobility vehicle is guided based on that the mobility vehicle enters the base plate and thus a position of the mobility vehicle is aligned with the center portion of the base plate.

3. The elevating apparatus of claim 2, wherein the base plate rotates after the position of the connection portion of the mobility vehicle is aligned with the center portion of the base plate by the contact portion so that the entrance/exit of the mobility vehicle or the traveling direction of the mobility vehicle is aligned with the at least one opening portion.

4. The elevating apparatus of claim 1, wherein in a state in which the mobility vehicle is aligned by the base plate, the multifunctional plate is matched with an external peripheral shape of the mobility vehicle or formed to be greater than the external peripheral shape.

5. The elevating apparatus of claim 1, further comprising:
a first lighting portion disposed on an upper surface of the multifunctional plate,
wherein the first lighting portion is turned on and implements a lighting function based on that the multifunctional plate moves to a lower side of the accommodation space.

6. The elevating apparatus of claim 1, further comprising:
a second lighting portion disposed on a lower surface of the multifunctional plate,
wherein the second lighting portion is turned on and implements a lighting function based on that the multifunctional plate moves to an upper side of the accommodation space.

7. The elevating apparatus of claim 1, further comprising:
a third lighting portion disposed at an upper side of the accommodation space of the elevator,
wherein the third lighting portion is turned on and implements a lighting function based on that the multifunctional plate moves to a lower side of the accommodation space.

8. The elevating apparatus of claim 1, further comprising:
a vehicle washing portion disposed on the multifunctional plate and configured to wash the mobility vehicle,
wherein the vehicle washing portion provides a vehicle washing function while operating based on that a door of the elevator and the entrance/exit of the mobility vehicle are closed.

9. The elevating apparatus of claim 8, wherein the multifunctional plate moves upward or downward together with the vehicle washing portion based on that the vehicle washing function is provided.

10. The elevating apparatus of claim 1, further comprising:
a first sterilization portion disposed on the multifunctional plate,
wherein the first sterilization portion is configured to sterilize the accommodation space of the elevator.

11. The elevating apparatus of claim 10, wherein the first sterilization portion operates after the passenger completely gets out of the accommodation space of the elevator.

12. The elevating apparatus of claim 1, further comprising:
a second sterilization portion disposed at an upper side of the accommodation space of the elevator,
wherein the second sterilization portion sterilizes the multifunctional plate.

13. The elevating apparatus of claim 1, wherein the multifunctional plate further includes a cleaning portion configured to draw foreign substances existing on a bottom portion of the accommodation space and the base plate based on that the multifunctional plate moves downward, and the cleaning portion provides a cleaning function.

14. The elevating apparatus of claim 1, further comprising:
at least one sensing portion disposed on the multifunctional plate,
wherein the at least one sensing portion measures a distance between the multifunctional plate and the mobility vehicle and identifies a position at which the multifunctional plate and the mobility vehicle do not interfere with each other.

15. The elevating apparatus of claim 14, wherein the base plate aligns a rotation position thereof based on information inputted through the at least one sensing portion so that the mobility vehicle and the multifunctional plate do not interfere with each other.

16. An elevating system comprising:
the elevating apparatus of claim 1, further including a contact portion disposed at a center portion of the base plate; and
the mobility vehicle including a connection portion extending so that a width of the connection portion decreases from a lower rim of the mobility vehicle toward a center portion of the mobility vehicle,
wherein the contact portion and the connection portion guide the mobility vehicle so that the mobility vehicle is positioned at the center portion of the base plate based on that the mobility vehicle enters the base plate.

17. The elevating apparatus of claim 16, wherein a door portion is disposed in an inlet of the connection portion of the mobility vehicle, and the door portion is opened based on that the mobility vehicle enters the base plate.

18. The elevating apparatus of claim 16,
wherein the mobility vehicle includes at least one sensor,
wherein based on that the mobility vehicle enters the base plate, the at least one sensor recognizes a position of the contact portion, and the connection portion moves toward the contact portion as the mobility vehicle enters the base plate to align with the contact portion.

19. A building including an elevating apparatus for a mobility vehicle, the building comprising:
a structure configured as a multi-storied structure and including a passageway configured to connect floors, and at least one opening/closing portion;
an elevator configured to be movable upward or downward along the passageway of the structure and including an accommodation space configured to accommodate the mobility vehicle, and at least one opening portion;
a base plate disposed in the elevator, configured to be rotatable on a bottom surface of the accommodation space, and configured to align an entrance/exit of the mobility vehicle and the at least one opening portion while rotating in a state in which the mobility vehicle is loaded on the base plate; and
a multifunctional plate disposed in the elevator and configured to move in an upward/downward direction of the structure in the accommodation space, the multifunctional plate being configured to cover the base plate between the entrance/exit of the mobility vehicle and the at least one opening portion of the elevator while moving downward to provide a foothold function to a passenger.

20. The building of claim 19, wherein based on that the elevator moves to a floor level at which the passenger gets out of the mobility vehicle or the mobility vehicle exits, the at least one opening/closing portion of the structure is opened after the multifunctional plate moves to a lower side of the elevator in a state in which the entrance/exit of the mobility vehicle and the at least one opening portion are aligned by the base plate.
